(19) United States
(12) Patent Application Publication
(10) Pub. No.: US 2013/0184490 A1
(43) Pub. Date: Jul. 18, 2013

(54) PROCESS TO PREPARE S-2-HYDROXY-3-METHOXY-3,3-DIPHENYL PROPIONIC ACID

(71) Applicants: Amala Kompella, Hyderabad (IN); Kali Satya Bhujanga Rao Adibhatla, Hyderabad (IN); Veera Swamy Balina, Hyderabad (IN); Srinivasu Kasa, Hyderabad (IN); Venkaiah Chowdary Nannapaneni, Hyderabad (IN)

(72) Inventors: Amala Kompella, Hyderabad (IN); Kali Satya Bhujanga Rao Adibhatla, Hyderabad (IN); Veera Swamy Balina, Hyderabad (IN); Srinivasu Kasa, Hyderabad (IN); Venkaiah Chowdary Nannapaneni, Hyderabad (IN)

(73) Assignee: NATCO PHARMA LIMITED, HYDERABAD (IN)

(21) Appl. No.: 13/814,038

(22) PCT Filed: Aug. 4, 2010
(86) PCT No.: PCT/IN10/00519

§ 371 Date: Mar. 25, 2013

(51) Int. Cl. C07C 51/09

(52) U.S. Cl. CPC C07C 51/09
USPC 562/468

(57) ABSTRACT

Disclosed is a process for the preparation of S-2-Hydroxy-3-methoxy-3,3-diphenylpropionic acid (I) the key intermediate for the preparation of Ambrisentan [(+)-2(S)-(4,6-Dimethylpyrimidin-2-yloxy)-3-methoxy-3,3-diphenylpropionic acid]. Ambrisentan of the formula (IA) is approved under the trademark “Letairis ®” by the US Food and Drug Administration for the treatment of Pulmonary artery hypertension (PAH).

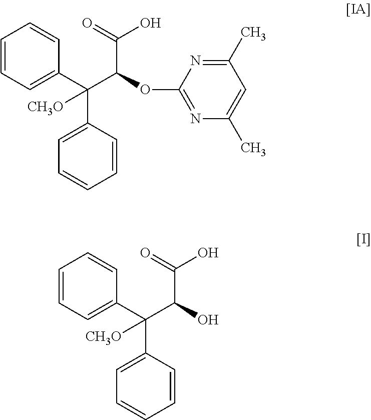

### BACKGROUND OF INVENTION

The preparation of S-2-Hydroxy-3-methoxy-3,3-diphenylpropionic acid is described in WO 9611914; eidem, U.S. Pat. No. 5,932,730 (1996, 1998 both to BASF) and in J. Med. Chem., 1996, vol. 39, No. 11, p. no. 2123-2128

In WO9611914 the following route is described (Scheme-1):

### Scheme-I

### Stage-I (Preparation of S-2-Hydroxy-3-methoxy-3,3-diphenylpropionic acid From Benzophenone)

Benzophenone is reacted with methyl chloroacetate in presence of sodium methoxide base. The product after work up is treated with p-toluene sulfonic acid and is hydrolysed with sodium hydroxide solution to yield 2-Hydroxy-3-methoxy -3,3-diphenyl propionic acid

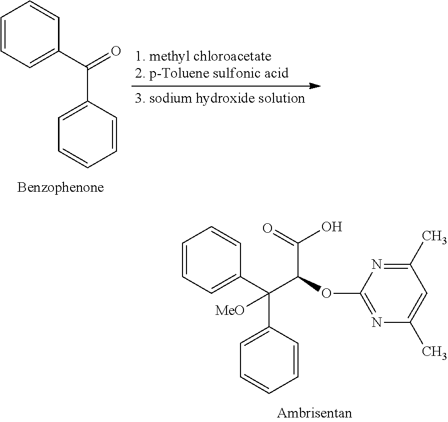 [SMILES: O=C(c1ccccc1)c1ccccc1 ; COC(c1ccccc1)(c1ccccc1)[C@H](Oc1nc(C)cc(C)n1)C(=O)O]

### Stage-II (Preparation of S-2-hydroxy-3-methoxy-3,3 diphenyl propionic acid of the Formula (I)

(S)-1-(4-nitrophenyl)ethylamine is added to 2-hydroxy-3-methoxy-3,3-diphenylpropionic acid in acetone and methyl tert-butyl ether under reflux, the mixture was seeded, boiled under reflux for one hour after cooling to RT The crystals (S-2-hydroxy-3-methoxy-3,3-diphenylpropionic acid salt of (S)-1-(4-nitrophenyl)ethylamine were filtered off. The salt is acidified and liberated compound of formula (I) is extracted into solvent. Solvent is distilled off to give compound of formula (I)

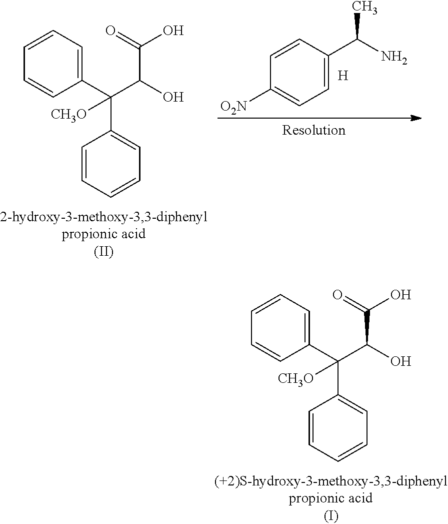

The following are the observations after implementing the above mentioned process in lab

### Stage-I

Step-I is one pot reaction involving three reactions epoxidation, rearrangement and hydrolysis. It does not yield pure product and is contaminated with all the starting materials and byproducts and number of purification steps are required to get pure product.

### Stage-II

This resolution stage is reported with resolving agents L-proline methyl ester and (S)-1-(4-nitrophenyl)ethylamine. L-proline methyl ester has not been worked out for resolution. After resolution with (S)-1-(4-nitrophenyl)ethylamine the yield of S-2-hydroxy-3-methoxy-3,3-diphenylpropionic acid is 15% with 0.5% enantiomeric impurity.

In the procedure discussed in J. Med. Chem., 1996, vol. 39, No. 11, stage-I is split into three steps as shown below (Scheme-II). In this paper the resolution of 2-hydroxy-3-methoxy-3,3-diphenyl propionic acid (II) is not mentioned.

### Scheme-II

### Step-I

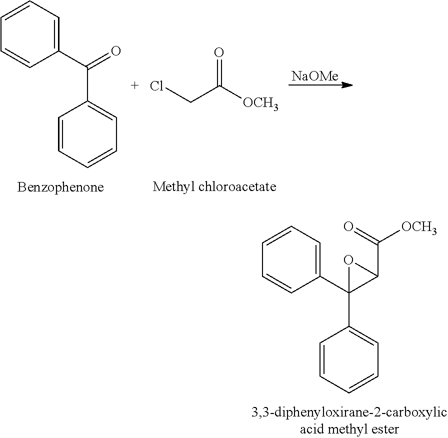 [SMILES: O=C(c1ccccc1)c1ccccc1 ; COC(=O)CCl ; COC(=O)C1OC1(c1ccccc1)c1ccccc1 ; "NaOMe"]

### Step-2

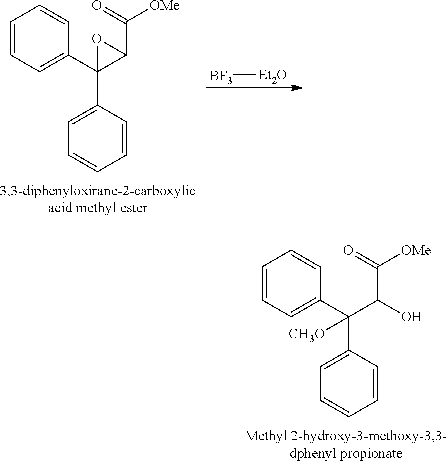

### Step-3

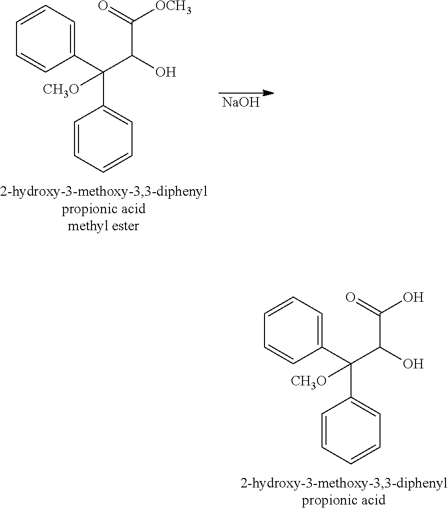 [0012] The above process involves borontrifluoride etherate in step-2 which is hazardous and flammable.
In step-3 solvent dioxane is used along with sodium hydroxide solution for hydrolysis. Dioxane is classified as class-1 solvent as per ICH guidelines and its usage is restricted.

### SUMMARY OF INVENTION

Taking into consideration the above mentioned shortcomings of the preparation of the compound of the formula I, our aim was directed towards developing an improved environmentally safe and industrially applicable process, which is devoid of the insufficiencies of the known method synthesis of pure desired compound (I) in high yields.

Therefore the main objective of the present invention is to provide an improved process for the preparation of Ambrisentan precursor S-2-hydroxy-3-methoxy-3,3-diphenylpropionic acid of the formula (I) avoiding the drawbacks of the hitherto known processes.

Another objective of the present invention is to provide an improved process for the preparation of Methyl 3,3-diphenyl 2,3-epoxy propionate of the formula (II)

Yet another objective of the present invention is to provide an improved process for the preparation of methyl-2-hydroxy-3-methoxy-3,3-diphenyl propionate of the formula (III)

Still another objective of the present invention is to provide an improved process for the preparation of 2-Hydroxy-3-methoxy-3,3-diphenyl propionic acid of the formula (IV)
Accordingly following scheme-3 illustrates current invention.

### Scheme-3

### Step-1: Preparation of (Methyl 3,3-diphenyl 2,3-epoxy propionate) of the Formula (II)

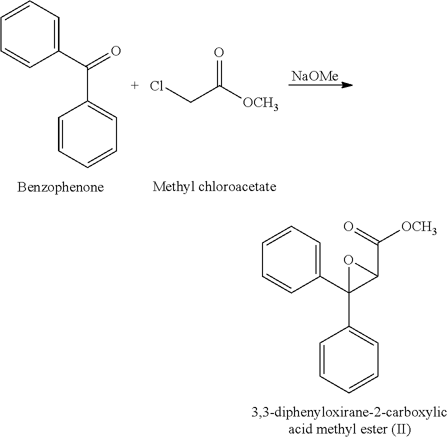 [SMILES: O=C(c1ccccc1)c1ccccc1 ; COC(=O)CCl ; COC(=O)C1OC1(c1ccccc1)c1ccccc1 ; "NaOMe"]

### Step-2 (Preparation of methyl-2-hydroxy-3-methoxy-3,3-diphenyl propionate) of the Formula (III)

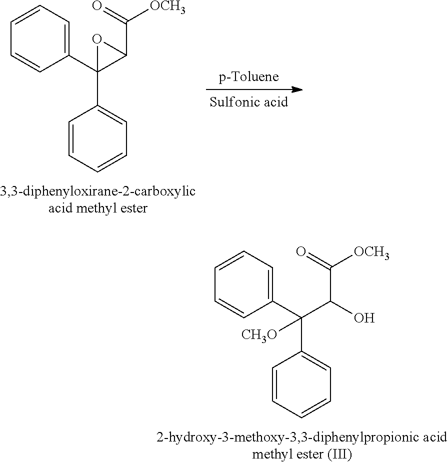

### Step-3: Preparation of 2-Hydroxy-3-methoxy-3,3-diphenyl propionic acid of the Formula (IV

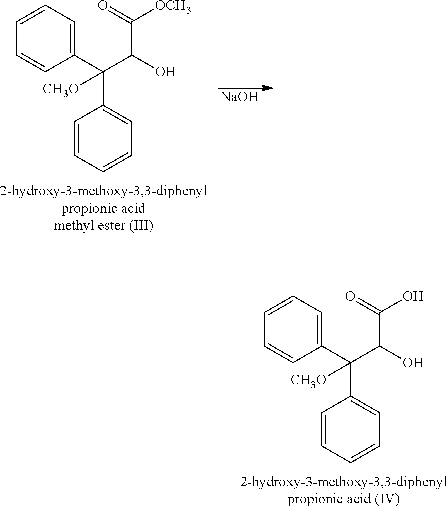

### Step-4: Preparation of S-2-hydroxy-3-methoxy-3,3-diphenyl propionic acid of the Formula (I)

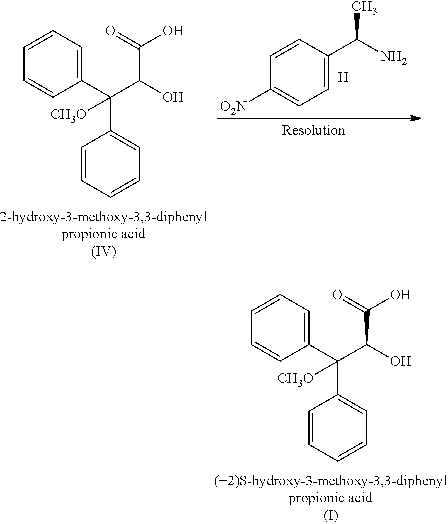

Accordingly, the present invention provides an improved method for the preparation of Methyl 3,3-diphenyl 2,3-epoxy propionate of the formula-(II)—which comprises
Charging Toluene into the flask
Charging benzophenone and stirring for 15 minutes
Charging sodium methoxide and stirring for 15 minutes
Cooling reaction mass to −10° to −5° C.
Slowly adding methyl chloroacetate at the same temperature
Maintaining reaction mass at −10° C. to −5° C. for one hour
Charging water and stirring for 30 minutes
Separating organic layer and washing with DM water
Distilling toluene completely under vacuum
Taking compound of Formula (II) next stage

Accordingly, the present invention provides an improved method for the preparation of methyl-2-hydroxy-3-methoxy-3,3-diphenyl propionate of the formula-(III) which comprises
Charging methanol to the compound of formula (II) and stirring for 30 minutes
Dissolving p-Toluene sulphonic acid monohydrate in methanol and adding slowly at 25-55° C.
Bringing reaction mass to room temperature and maintaining for one hour.
Cooling reaction mass to 0-5° C. and maintaining at the same temperature for 2 hours
Filtering and washing with methanol
Dissolving wet compound in ethyl acetate and washing with 5% sodium bicarbonate solution.
distilling off completely under vacuum.
Bringing residue to room temperature and charging n-hexane.
Maintaining under stirring at room temperature for 2 hours.
Filtering and washing with n-hexane to yield compound of formula (III)

Accordingly, the present invention provides an improved method for the preparation of 2-hydroxy-3-methoxy-3,3-diphenyl propionic acid of the formula-(IV) which comprises
Charging purified water into flask
Charging compound of formula (III) and stirring for 10 minutes.
Charging 1N sodium hydroxide solution and stirring for 15 minutes.
Heating reaction mass to 90-95° C.
Maintaining one hour at the same temperature
Adjusting reaction mass pH to 2-3 with 1N hydrochloric acid
Cooling reaction mass to 5-10° C.
Maintaining reaction mass at the same temperature for 2 hours.
Filtering to yield compound of formula (IV)

Accordingly, the present invention provides an improved method for the preparation of S-2-hydroxy-3-methoxy-3,3-diphenyl propionic acid of the formula-(I) which comprises
Charging Methyl t-butyl ether (MTB) and acetone into flask
Charging compound of formula (IV) and stirring for 15 minutes
Heating reaction mass to reflux temperature (55° C.)
Dissolving S-(-)p-nitro phenyl ethyl amine in methyl tert-butyl ether (MTB) and adding slowly during 30 minutes at reflux temperature
Maintaining reaction mass under reflux temperature for one hour.
Bringing reaction mass slowly to room temperature during 2 hours.
Cooling reaction mass to 10-15° C.
Maintaining reaction mass at the same temperature for 12 hours.
Filtering and washing with MTB.
Suspending wet salt in a mixture of DM water and MTB.
Acidifying reaction mass with concentrated hydrochloric acid
Stirring reaction mass for 30 minutes and separating two clear layers.
Extracting aqueous layer with MTB and combining organic layers.
washing MTB layer with water and drying over sodium sulphate
Distilling off MTB completely and charging MTB and n-heptane to the residue at 40° C.
Bringing residue to room temperature
Stirring at room temperature for 2-3 hours.
filtering and washing with n-Heptane to yield compound of formula (I)

The solid state properties of S-2-hydroxy-3-methoxy-3,3-diphenyl propionic acid of the formula-(I) thus prepared are illustrated by the following figures:

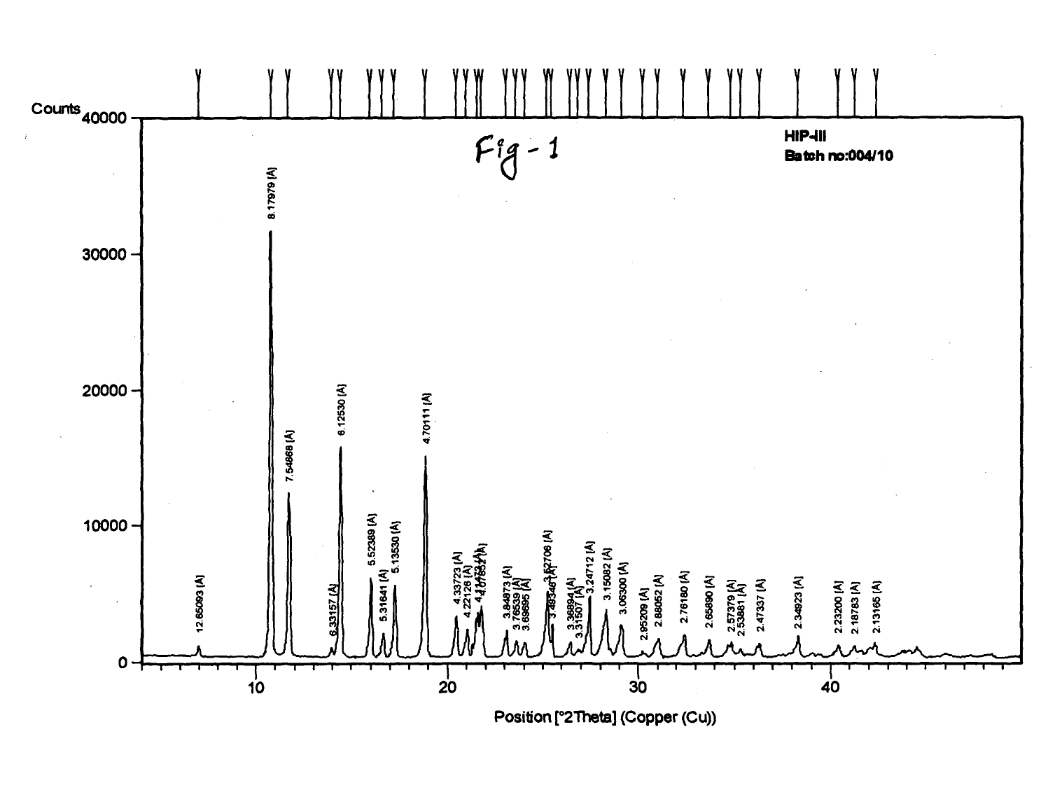

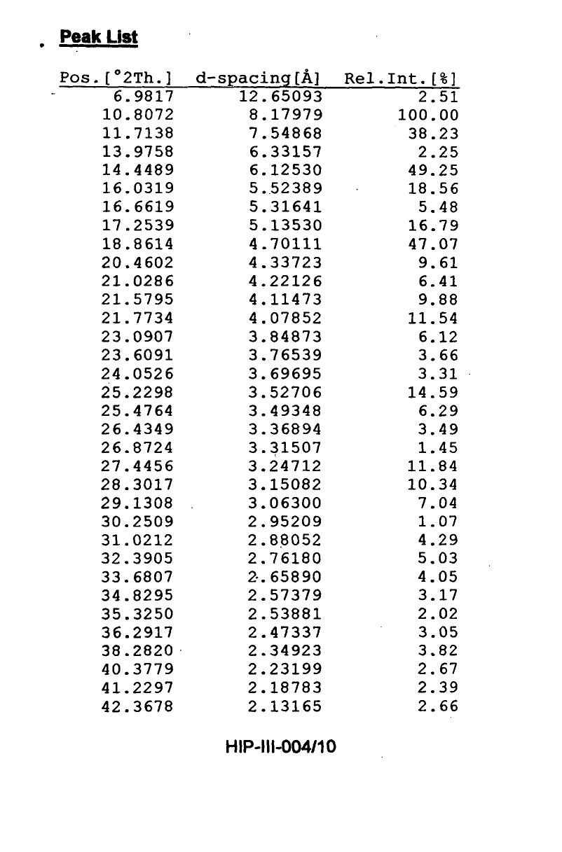

FIG. 1—XRPD spectrum of the compound of the formula-I prepared by the method disclosed in example-1

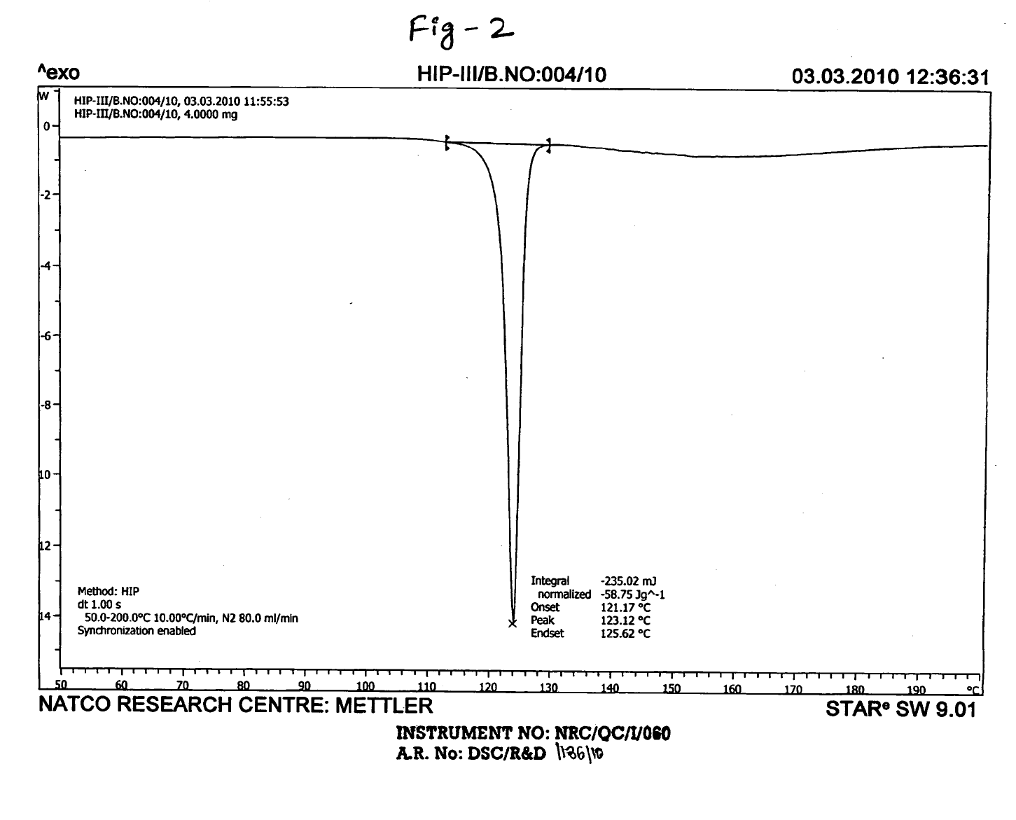

FIG. 2—DSC curve of the compound of the formula-I prepared by the method disclosed in example-1

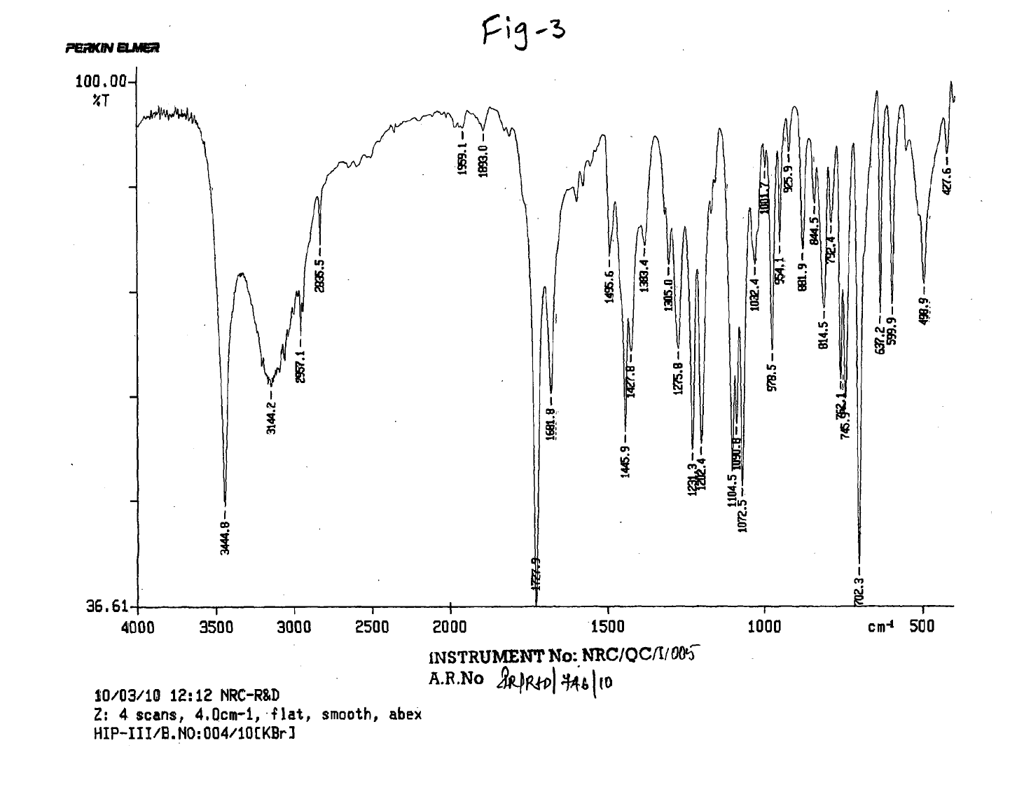

FIG. 3—IR spectrum of the compound of the formula-I prepared by the method disclosed in example-1

The details of the inventions are given in the Examples which are provided for illustration only and therefore the Examples should not be construed to limit the scope of the invention.

### EXAMPLE

Process for the Preparation S-2-Hydroxy-3-methoxy-3,3-diphenyl propionic acid of the Formula-I

### Step-1: Preparation of Methyl 3,3-diphenyl 2,3-epoxy propionate(II)

Into a 5 L round bottomed flask a mixture of toluene (1.2 L) and benzophenone (500 g) were charged and stirred for 15 minutes. Sodium methoxide (260 g) was charged and the reaction mixture stirred for 15 minutes. Reaction mass was cooled to −5 to −10° C. and methyl chloro acetate (506.3 g dissolved in 0.3 L toluene) was added slowly during 90 minutes at the same temperature. Reaction mass was maintained at the same temperature and purified water (1 L) was added. Reaction mass was stirred for 1 hour and toluene layer was separated. Toluene layer was distilled of completely under vacuum. The residual compound II was taken for next step.

Yield: 660 g (94.8%)

Purity by HPLC: 95.5% (benzophenone content: 4%)

### Step-II: Preparation of methyl-2-hydroxy-3-methoxy-3,3-diphenyl propionate (III)

Into a 5 L round bottomed flask a mixture of methanol (1.2 L) and compound of formula-I (660 g) from the previous step were charged and stirred for 15 minutes. p-Toluene sulphonic acid (15 g dissolved in 150 ml methanol) was slowly added during 30 minutes at 25-55° C. The reaction mass was brought to room temperature, maintained at the same temperature for 2 hours was filtered and the filtered compound was dissolved in ethyl acetate (3 L). Ethyl acetate layer was washed with 5% sodium bicarbonate solution (2×1 L). Ethyl acetate layer was distilled off completely under vacuum. To the residue hexane (1L) was charged and maintained under stirring for 2 hours. The product was filtered and dried at 50-60° C.

Dry weight: 560 g (75%)

Purity by HPLC: 99.9%

Melting range: 100-102° C.

### Step-III: Preparation of 2-hydroxy-3-methoxy-3,3-diphenyl propionic acid of the Formula-(IV)

Into a 5 L round bottomed flask a mixture water (1.0 L) and compound of formula-III (200 g) from step-II were charged and stirred for 15 minutes. 1N aqueous sodium hydroxide solution was charged and the reaction mass was stirred for 15 minutes. Reaction mass was heated to 90-95° C. and maintained at the same temperature for one hour. The reaction mass was brought to room temperature adjustment of pH was carried out with 1N hydrochloric acid solution (1.6 L) to 2-3. The product slurry was cooled to 5-10° C. and maintained at the same temperature for 2 hours. The product was filtered and dried at 60-65° C.

Dry weight: 172 g (90%)

Purity by HPLC: 99.88%

Melting range: 100-102° C.

### Step-IV: Preparation of S-2-hydroxy-3-methoxy-3,3-diphenyl propionic acid of the Formula-(I)

Into a 3 L round bottomed flask compound of formula (IV) from step-III (200 g) was added to a mixture methyl tert-butyl ether (1.25 L) and acetone (1.5 L). Reaction mass was heated to reflux temperature (50-55° C.) and S-(-)p-nitro phenyl ethyl amine (61 g dissolved in 250 ml of MTB) was added slowly during 30 minutes at reflux temperature. Reaction mass was maintained at the same temperature for one hour and cooled to 10-15° C. and maintained at the same temperature for 12 hours. It was filtered and washed with MTB (500 ml). The wet diastereomeric salt was suspended in a mixture of MTB (500 ml) and water (1.5 L) and acidified with concentrated hydrochloric acid (30 ml). the resulting mixture was stirred for 30 minutes and the organic layer was separated.

Aqueous layer was extracted with MTB (500 ml) and the combined MTB layer was washed with water and distilled completely under vacuum. Reaction mass was brought to 40° C. and mixture of MTB (138 ml) and n-heptane (322 ml) were charged and the cooled to 25-30° C. The crystalline compound of formula-I was filtered and dried at 50-60° C.

Dry weight: 55 g (55%)

Purity by HPLC: 99.97% (chemical purity)
99.98% (chiral purity)

Melting range: 123-125° C.

### Advantages of the Invention

1) S-2-Hydroxy-3-methoxy-3,3-diphenyl propionic acid is produced in more than 99.8% chemical purity.

2) The chiral purity of S-2-Hydroxy-3-methoxy-3,3-diphenyl propionic acid by the process of the present invention is about 99.9%

3) S-2-Hydroxy-3-methoxy-3,3-diphenyl propionic acid prepared by this method is suitable for synthesis of pharmaceutical grade Ambrisentan.

1. improved process for the preparation of S-2-hydroxy-3-methoxy-3,3-diphenylpropionic acid (I) 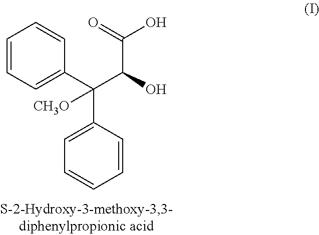
comprising the steps:
a) Reacting the benzophenone with methyl chloroacetate to get the compound of formula II 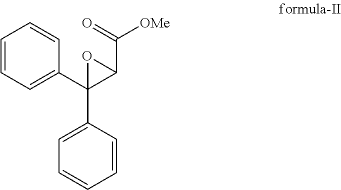 [SMILES: COC(=O)C1OC1(c1ccccc1)c1ccccc1]
b) Reacting the compound of formula (II) with p-toluene sulphonic acid to get the compound of formula (III). 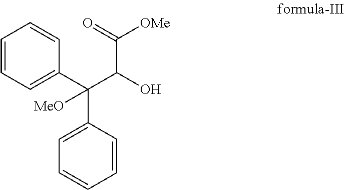 [SMILES: COC(=O)C(O)C(OC)(c1ccccc1)c1ccccc1]
c) Hydrolysing the compound of formula III with sodium hydroxide to get the compound of formula (IV). 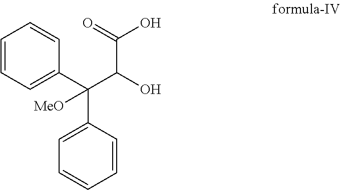 [SMILES: COC(c1ccccc1)(c1ccccc1)C(O)C(=O)O]
d) Resolving the compound of formula (IV) by reacting it with S-(-)p-nitro phenyl ethyl amine dissolved in methyl tert-butyl ether to get the compound of formula-I.

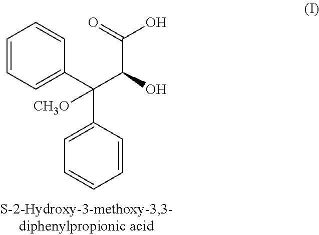

2. The process as claimed in claim 1 wherein the step (a) comprises:
(i) Charging Toluene into the flask
(ii) Charging benzophenone and stirring for 15 minutes
(iii) Charging sodium methoxide and stirring for 15 minutes
(iv) Cooling reaction mass to −10° to −5° C.
(v) Slowly adding methyl chloroacetate at the same temperature
(vi) Maintaining reaction mass at −10° C. to −5° C. for one hour
(vii) Charging water and stirring for 30 minutes
(viii) Separating organic layer and washing with DM water
(ix) Distilling toluene completely under vacuum
(x) Taking compound of Formula (II) to next stage

3. The process as claimed in claim 1 wherein the step (b) comprises:
(i) Charging methanol to the compound of formula (II) and stirring for 30 minutes
(ii) Dissolving p-Toluene sulphonic acid monohydrate in methanol and adding slowly at 25-55° C.
(iii) Bringing reaction mass to room temperature and maintaining for one hour.
(iv) Cooling reaction mass to 0-5° C. and maintaining at the same temperature for 2 hours
(v) Filtering and washing with methanol
(vi) Dissolving wet compound in ethyl acetate and washing with 5% sodium bicarbonate solution.
(vii) distilling off completely under vacuum.
(viii) Bringing residue to room temperature and charging n-hexane.
(ix) Maintaining under stirring at room temperature for 2 hours.
(x) Filtering and washing with n-hexane to yield compound of formula (III)

4. The process as claimed in claim 1 wherein the step (c) comprises:
(i) Charging purified water into flask
(ii) Charging compound of formula (III) and stirring for 10 minutes.
(iii) Charging IN sodium hydroxide solution and stirring for 15 minutes.
(iv) Heating reaction mass to 90-95° C.
(v) Maintaining one hour at the same temperature
(vi) Adjusting reaction mass pH to 2-3 with 1N hydrochloric acid
(vii) Cooling reaction mass to 5-10° C.
(viii) Maintaining reaction mass at the same temperature for 2 hours.
(ix) Filtering to yield compound of formula (IV)

5. The process as claimed in claim 1 wherein the step (d) comprises:
(i) Charging Methyl t-butyl ether (MTB) and acetone into flask
(ii) Charging compound of formula (IV) and stirring for 15 minutes
(iii) Heating reaction mass to reflux temperature (55° C.)
(iv) Dissolving S-(-)p-nitro phenyl ethyl amine in methyl tert-butyl ether (MTB) and adding slowly during 30 minutes at reflux temperature
(v) Maintaining reaction mass under reflux temperature for one hour.
(vi) Bringing reaction mass slowly to room temperature during 2 hours.
(vii) Cooling reaction mass to 10-15° C.
(viii) Maintaining reaction mass at the same temperature for 12 hours.
(ix) Filtering and washing with MTB.
(x) Suspending wet salt in a mixture of DM water and MTB.
(xi) Acidifying reaction mass with concentrated hydrochloric acid
(xii) Stirring reaction mass for 30 minutes and separating two clear layers.
(xiii) Extracting aqueous layer with MTB and combining organic layers.
(xiv) washing MTB layer with water and drying over sodium sulphate
(xv) Distilling off MTB completely and charging MTB and n-heptane to the residue at 40° C.
(xvi) Bringing residue to room temperature
(xvii) Stirring at room temperature for 2-3 hours.
(xviii) filtering and washing with n-Heptane to yield compound of formula (I)

6. A method of preparing the S-2-hydroxy-3-methoxy-3,3-diphenyl propionic acid of formula (I) essentially as described in example-1.

7. A method of preparing S-2-hydroxy-3-methoxy-3,3-diphenyl propionic acid of formula (I) as claimed in claim 1 and having chemical purity of more than 99.8% and chiral purity of more than 99.9%.

8. A method of preparing S-2-hydroxy-3-methoxy-3,3-diphenyl propionic acid of formula (I) as claimed in claim 1 and having solid state characteristics as in FIGS. 1-3.
